(19) United States
(12) Patent Application Publication
(10) Pub. No.: US 2003/0099619 A1
(43) Pub. Date: May 29, 2003

(54) Method and composition for targeting an adenoviral vector

(75) Inventors: Thomas J. Wickham, Germantown, MD (US); Imre Kovesdi, Rockville, MD (US); Petrus W. Roelvink, Germantown, MD (US); David Einfeld, Germantown, MD (US); Douglas E. Brough, Gaithersburg, MD (US); Alena Lizonova, Gaithersburg, MD (US)

Correspondence Address: LEYDIG VOIT & MAYER, LTD, TWO PRUDENTIAL PLAZA, SUITE 4900, 180 NORTH STETSON AVENUE, CHICAGO, IL, 60601-6780, US

(73) Assignee: GenVec, Inc., Gaithersburg, MD

(21) Appl. No.: 10/304,160

(22) Filed: Nov. 25, 2002

Related U.S. Application Data
(63) Continuation of application No. PCT/US01/17391, filed on May 30, 2001.
Application No. PCT/US01/17391 is a continuation-in-part of application No. 09/631,191, filed on Aug. 2, 2000.
(60) Provisional application No. 60/208,451, filed on May 31, 2000.

(51) Int. Cl. A61K 48/00; C12Q 1/70; C12N 15/861; C07K 14/705; C07K 14/075

(52) U.S. Cl. 424/93.2; 435/5; 435/456; 435/235.1; 435/325; 530/350

(57) ABSTRACT

The invention provides adenoviral coat proteins comprising various non-native ligands. Further, the present invention provides an adenoviral vector that elicits less reticulo-endothelial system (RES) clearance in a host animal than a corresponding wild-type adenovirus. Also provided by the invention is a system comprising a cell having a non-native cell-surface receptor and a virus having a non-native ligand, wherein the non-native ligand of the virus binds the non-native cell-surface receptor of the cell. Using this system, a virus can be propagated. Further provided by the invention is a method of controlled gene expression utilizing selectively replication competence, a method of assaying for gene function, a method of isolating a nucleic acid, and a method of identifying functionally related coding sequences. Additionally, the invention provides a cell-surface receptor, which facilitates internalization.

### CROSS-REFERENCE TO RELATED PATENT APPLICATIONS

This patent application is a continuation of International Patent Application No. PCT/US01/17391, filed May 30, 2001, which designates the U.S., and which is a continuation-in-part of U.S. patent application Ser. No. 09/631,191, filed Aug. 2, 2000, which claims the benefit of U.S. Provisional Patent Application No. 60/208,451, filed May 31, 2000.

### TECHNICAL FIELD OF THE INVENTION

This invention pertains to methods and compositions useful in targeting adenoviral vectors.

### BACKGROUND OF THE INVENTION

Viral vectors, and particularly adenoviruses, can be engineered into gene transfer vectors, such as for use in gene therapy applications and basic animal research. For in vivo applications, it is often desirable to contain transgene expression within discrete tissue types or organs. For example, contained gene expression would facilitate investigation of the effects of in vivo gene transfer within predefined tissue types, thus minimizing background or nonspecific activities in other tissue types. Of course, ectopic expression presents itself as a major hurdle for many potential clinical protocols. One strategy for containing vector-based gene expression is to employ tissue specific promoters to drive transgene expression. Another approach is to alter viral tropism, such that the vector binds to the desired tissue or organ types with far more affinity than other tissues, an approach referred to as “targeting” (see, e.g., U.S. Pat. Nos. 5,559,099; 5,712,136; 5,731,190; 5,770,440; 5,871,726; and 5,830,686 and International Patent Applications WO 96/07734, WO 98/07877, WO 97/07865, WO 98/54346, WO 96/26281, and WO 98/40509). While such technology is known generally, there remains a need for adenoviruses that are targeted to specific tissue types and, indeed, identified cell surface proteins.

Related to the problem of viral targeting is replication of alternatively targeted vectors. Viruses lacking native tropism generally are unable to productively infect the cells typically employed to replicate them. While pseudo-receptor cell lines have been developed (see, e.g., International Patent Application WO 00/14269), it would be desirable to better ensure that viruses are able to not only bind such cell proteins, but also to be internalized into the producer cells efficiently. Thus, there remains a need for improved cell lines able to replicate alternatively targeted vectors.

Along with ectopic gene expression, destruction and clearance of gene therapy vectors stands as another obstacle to in vivo use of many adenoviral based gene therapy vectors. For example, adenoviral coat proteins, particularly the hexon, contain antigenic motifs that readily alert a healthy immune system. Additionally, adenoviruses are actively scavenged from circulation by cells of the reticulo-endothelial system (RES) (see, e.g., Worgall et al., Hum Gene Ther., 8, 1675-84 (1997); Wolff et al., J. Virol., 71(1), 624-29 (1997)). In such a response, Kupffer cells, endothelial liver cells, or other RES cells scavenge the virus from the circulation (see generally, Moghini et al., Crit. Rev. Ther. Drug Carrier Sys., 11(1), 31-59 (1994); Van Rooijen et al., J. Leuk. Biol., 62, 702-09 (1997)). For example, virus can become opsonized, possibly though interaction between collections and glycosylated viral proteins, triggering recognition by RES cells; alternatively, RES cells can recognize charged amino acid residues on the virion surface (see Hansen et al., Immunobiol, 199(2), 165-89 (1998); Jahrling et al., J. Med. Virol., 12(1), 1-16 (1983)). Such interactions lead to destruction of viral vectors, thereby reducing the effective free titer of the vectors and their half-life. Some existing technology for reducing immunogenicity involves mutating viral coat proteins, particularly the hexon protein, to reduce viral interaction with neutralizing antibodies (see, e.g., U.S. Pat. No. 6,612,525 and International Patent Application WO 98/40509). However, this approach does not appear to effectively mitigate viral clearance by the RES. Thus, there remains a need for more stealthy vectors—i.e., those able to avoid host defenses.

In the treatment of various diseases, often it is advantageous to determine the exact location of disease tissue. Upon systemic administration of a treatment via an adenoviral vector targeted to specific tissue, a clinician does not necessarily know the precise location of the cells that are targeted by the adenoviral vector. One such situation is treatment of a tumor. It would be helpful if an initial anti-tumor agent is administered in combination with an agent that allows a clinician to image the affected cells. Such technology would allow the clinician to direct any additional treatment directly to those cells where it is needed.

### BRIEF SUMMARY OF THE INVENTION

The invention provides an adenoviral coat protein comprising various non-native amino acid sequences. The adenoviral coat protein can comprise a non-native ligand, wherein the non-native ligand binds to a substrate selected from the group of substrates consisting of melanocortin receptor (MC1), αv integrins, αvβ3 integrin, αvβ6 integrin, α4 integrins, α5 integrins, α6 integrins, α9 integrins, CD13, melanoma proteoglycan, membrane dipeptidase (MDP), TAG72 antigen, an antigen binding site of a surface immunoglobulin receptor of B-cell lymphomas, type I interleukin I (IL-1) receptor, human immunodeficiency virus type 1 (HIV-1) envelope glycoprotein (gp120), atrial natriuretic peptide (ANP) receptor, erythropoietin (EPO) receptor, thrombopoietin (TPO) receptor, carcino-embryonic antigen (CEA) receptor, EpCAM, CD40, prostate-specific membrane antigen (PSMA), endoglin (CD105), epidermal growth factor receptor (EGFR), HER2, and extracellular matrix components, such as collagen. The present invention further provides a recombinant adenoviral coat protein comprising a non-native ligand that binds to a substrate selected from the group consisting of α4 integrins, αv integrins, αvβ3 integrin, and αvβ6 integrin, wherein an adenoviral vector comprising the recombinant coat protein lacks native binding to coxsackievirus and adenovirus receptor (CAR). Also, the present invention provides an adenoviral vector comprising an adenoviral coat protein that elicits less reticulo-endothelial system (RES) clearance in a host animal than a corresponding wild-type adenovirus.

Also provided by the invention is a system comprising a cell having a non-native cell-surface receptor and a virus having a non-native ligand, wherein the non-native ligand of the virus binds the non-native cell-surface receptor of the cell. Preferably, the cell is in vivo. Using this system, a virus can be propagated. This method of propagation involves infecting a cell of the present inventive system with a virus, maintaining the cell, and recovering the virus produced within the cell. Alternatively, the present invention provides a method of propagating a virus comprising (a) infecting a cell having a non-adenovirus cell-surface receptor with a virus having a non-native ligand, wherein the non-native ligand binds the non-adenovirus cell-surface receptor. The method further comprises maintaining the cell and recovering the virus produced.

Also provided by the present invention is a method of assaying for gene function comprising (a) infecting a cell having a non-native cell-surface receptor with a gene transfer vector comprising a ligand that binds the non-native receptor of the cell, (b) maintaining the cell, and (c) assaying the cell for alterations in physiology. The present invention also provides a method of isolating a nucleic acid encoding a product comprising a desired property and a method of identifying functionally related coding sequences.

Further provided by the invention is a method of controlled gene expression, which is accomplished by administering to an animal a selectively replication competent adenoviral vector having a first non-native nucleic acid and a second non-native nucleic acid. Particularly, to accomplish controlled gene expression, the first non-native nucleic acid is for transcription and the second non-native nucleic acid is for selective replication.

Additionally, the invention provides a cell-surface receptor comprising a first domain and a second domain. The first domain of the present inventive cell-surface receptor binds an adenoviral vector having one or more chimeric adenoviral coat proteins and the second domain facilitates internalization of the adenoviral vector into a cell. The second domain also can be a chemical linkage to the cell membrane, such as a glycerol-phosphate-inositol (GPI) linkage. The present invention further provides a cell expressing the inventive cell-surface receptor.

Finally, the invention provides a method of therapy involving administration to an animal of an adenoviral vector having a first non-native nucleic acid and a second non-native nucleic acid. The first non-native nucleic acid specifically encodes a therapeutic agent and the second non-native nucleic acid encodes an agent that facilitates imaging.

The present invention is useful in a variety of applications, in vitro and in vivo, such as therapy, for example, as a vector for delivering a therapeutic gene to a cell with minimal ectopic infection. Specifically, the present invention permits more efficient production and construction of safer vectors for gene therapy applications. The present invention is also useful as a research tool by providing methods and reagents for the study of adenoviral attachment and infection of cells, assaying receptor-ligand interactions, and assaying for gene functions. Similarly, the recombinant adenoviral coat proteins can be used in receptor-ligand assays and as adhesion proteins in vitro or in vivo. Additionally, the present invention provides reagents and methods permitting biologists to investigate the cell biology of viral growth and infection. Thus, the inventive adenoviral vectors and methods are highly useful in biological research. These and other advantages of the present invention, as well as additional inventive features, will be apparent from the following detailed description and accompanying Sequence Listing.

### DETAILED DESCRIPTION OF THE INVENTION

Recombinant Adenoviral Vectors

The present invention provides a recombinant adenoviral coat protein comprising a non-native ligand. This non-native ligand binds to a substrate selected from the group of substrates consisting of melanocortin receptor (MCI), αv integrins, αvβ6 integrin, α4 integrins, α5 integrins, α6 integrins, α9 integrins, CD13, melanoma proteoglycan, membrane dipeptidase (MDP), TAG72 antigen, an antigen binding site of a surface immunoglobulin receptor of B-cell lymphomas, type I interleukin I (IL-1) receptor, human immunodeficiency virus type 1 (HIV-1) envelope glycoprotein (gp120), atrial natriuretic peptide (ANP) receptor, erythropoietin (EPO) receptor, thrombopoietin (TPO) receptor, carcino-embryonic antigen (CEA) receptor, EpCAM, CD40, PSMA, endoglin, EGFR, HER2 (otherwise known as erb B2) and an extracellular matrix component. Preferably, the extracellular matrix component is collagen. The adenoviral coat protein can comprise a non-native ligand that binds a substrate selected from the group of substrates consisting of αv integrins, αvβ3 integrin, and αvβ6 integrin. Suitable ligands for these substrates, which are conjugated to the adenoviral coat proteins, can, for example, comprise a sequence of amino acids selected from the group of sequences consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, and SEQ ID NO:31, as well as conservatively modified variants of these sequences. Ligands for these substrates are well known in the art and are described in various references (see, e.g., Szardenings at al., J. Bio. Chem., 272(44), 27, 943-48 (1997); Koivunen et al., J. Cell Biol., 124(3), 373-80 (1994); Pasqualini et al., Nat. Biotechnol., 15(6), 542-46 (1997); Kraft et al., J. Biol. Chem., 274(4), 1979-85 (1999); Vanderslice et al., J. Immunol., 158(4), 1710-18(1997); Koivunen et al., J. Cell Biol., 124(3), 373-80 (1994); Murayama et al., J. Biochem. (Tokyo), 120(2), 445-51(1996); Schneider et al., FEBS Lett., 429(3), 269-73 (1998); Pasqualini et al., Cancer Res., 60(3), 722-27 (2000); Arap et al., Science, 279(5349), 377-80 (1998); Burg et al., Cancer Res., 59(12), 2869-74 (1999); Rajotte and Ruoslahti, J. Biol. Chem., 274 (17), 11,593-98 (1999); Rajotte et al., J. Clin. Invest., 102(2), 430-37 (1998); Gui et al., Proteins, 24(3), 352-58 (1996); Gui et al., Biochem. Biophys. Res. Commun., 218(1), 414-19 (1996); Renschler et al., Proc. Nat'l Acad. Sci USA, 91(9), 3623-27 (1994); Yanofsky et al., Proc. Nat'l Acad. Sci. USA, 93(14), 7381-86 (1996); Ferrer and Harrison, J. Virol., 73(7), 5795-802 (1999); Herrmann et al., Biochim. Biophys. Acta, 1472(3), 529-36 (1999); Samoylova and Smith, Muscle Nerve, 22(4), 460-66 (1999); Li et al., Science, 270(5242), 1657-60 (1995); McConnell et al., Biol. Chem., 379(10), 1279-86 (1998); Cwirla et al., Science, 276(5319), 1696-99 (1997)); and Hall et al., Human Gene Therapy, 11, 983-993 (2000). The ligand is not limited to those sequences, or even conservative modifications of them. In this regard, other ligands can be identified using approaches described in the art for identifying peptide sequences that can act as ligands for a cell-surface receptor and, hence, are of use in the present invention (see, e.g., Russell, Nature Medicine, 2, 276-277 (1996)).

Preferably, the non-native ligand of the recombinant adenoviral coat protein allows an adenoviral vector comprising the coat protein to bind and, desirably, infect host cells not naturally infected by adenovirus (i.e., host cells not infected by wild-type adenovirus) or to bind to particular target cells with greater affinity than non-target cells. For example, the non-native ligand can preferentially bind a substrate, such as αvβ3 integrin, to target an adenoviral vector to cells displaying the integrin. To increase targeting efficiency, native binding of the adenoviral coat protein to native adenoviral cell-surface receptors, such as CAR, can be ablated. By “preferentially binds” is meant that the non-native ligand binds, for instance, αvβ3 integrin with about 3-fold greater affinity to about 50-fold greater affinity (e.g., 5-fold, 10-fold, 15-fold, 20-fold, 25-fold, 35-fold, or 45-fold greater affinity) than the non-native ligand binds αvβ1 integrin. Preferably, the non-native ligand binds αvβ3 integrin with about 10-fold greater affinity than it binds to αvβ1 integrin. Binding affinity can be determined using a variety of assays. For example, transduction levels of host cells are indicative of binding efficiency. Host cells displaying αvβ3 integrin on the cell surface (e.g., MDAMB435 cells) and host cells displaying predominantly αvβ1 on the cell surface (e.g., 293 cells) are exposed to wild type adenovirus and adenoviral vectors comprising the recombinant coat protein. Comparison of the transduction efficiencies points to relative binding affinity.

Adenoviral vectors displaying the ligands discussed herein are useful tools in therapeutics and research. For example, αvβ3 integrins are upregulated in tumor tissue vasculature, metastatic breast cancer, melanoma, and gliomas. Adenoviral vectors displaying ligands specific for αvβ3 integrin, such as an RGD motif, infect cells with a greater number of αvβ3 integrin moieties on the cell surface compared to cells that do not express the integrin to such a degree, thereby targeting the vectors to specific cells of interest. In addition, it appears that adenoviral vectors displaying the ligand for αvβ3 integrin and lack native binding have a longer half-life in serum compared to vectors where native binding was ablated and demonstrate decreased tropism to non-cancerous tissue, such as kidney and lung. Clearly the adenoviral vectors described herein are useful in a variety of therapeutic and research settings.

Alternatively, the recombinant coat protein can comprise a non-native ligand and a non-native amino acid sequence, wherein the non-native ligand binds to matrix metalloproteinase (MMP) and the non-native amino acid sequence comprises a chimeric adenoviral coat protein. The ligand for MMP, which is conjugated to the adenoviral coat protein, preferably comprises SEQ ID NO:12, as well as conservatively modified variants of this sequence. Both the MMP receptor and ligand are well know in the art (see, e.g., Koivunen et al., Nat. Biotechnol., 27(8), 768-74 (1999)).

The non-native ligand can be conjugated to any of the adenoviral coat proteins. Therefore, for example, the non-native ligand of the present invention can be conjugated to a fiber protein, a penton base protein, a hexon protein, proteins IX, VI, or IIIa, etc. Of course, the adenoviral coat protein can be derived from any of the adenoviral serotypes (e.g., serotypes 2 or 5). The sequences of such proteins, and methods for employing them in recombinant proteins, are well known in the art (see, e.g., U.S. Pat. Nos. 5,559,099; 5,712,136; 5,731,190; 5,770,442; 5,846,782; 5,962,311; 5,965,541; 5,846,782; and 6,057,155; and International Patent Applications WO 96/07734, WO 96/26281, WO 97/20051, WO 98/07877, WO 98/07865, WO 98/40509, WO 98/54346, and WO 00/15823). The coat protein portion of the inventive recombinant proteins can be a full-length adenoviral coat protein to which the ligand domain is appended, or it can be truncated, e.g., internally or at the C- and/or N-terminus. However modified (including the presence of the non-native amino acid), the inventive protein preferably is able to incorporate into an adenoviral capsid as its native counterpart coat protein.

Two or more of the adenoviral coat proteins are believed to mediate attachment to cell surfaces (e.g., fiber, penton base, etc.). Where such an adenoviral protein is employed within the inventive recombinant protein, it can further lack native binding. Any suitable technique for altering such binding can be employed. For example, exploiting differing fiber lengths to ablate native binding to cells can be accomplished via the addition of a binding sequence to the penton base or fiber knob. This addition can be done either directly or indirectly via a bispecific or multispecific binding sequence. In another embodiment, the fiber can be shortened (see, e.g., U.S. Pat. No. 5,962,311), or nucleic acid residues associated with native substrate binding can be mutated (see, e.g., International Patent Application PCT/US99/20728) such that it is less able to bind its native substrate (e.g., CAR), at least when incorporated into a mature virion. Similarly, the penton base can be mutated, e.g., by destroying the RGD sequence, to reduce its ability to bind integrin molecules.

The inventive recombinant coat protein can be constructed by any suitable method. Preferably the recombinant adenovirus coat protein comprises a non-native ligand wherein the alteration is made at the level of DNA. Methods of DNA manipulation (e.g., additions, deletions, substitutions, creation of fusion proteins, etc.) are well known in the art (see, for instance, Sambrook et al., Molecular Cloning, A Laboratory Manual, 2d edition, Cold Spring Harbor Press, Cold Spring Harbor, N.Y. (1989)). The nucleic acid sequence encoding the non-native ligand can be inserted into or in place of an internal coat protein sequence, or, alternatively, the non-native amino acid sequence can be at or near the C-terminus or N-terminus of the chimeric adenovirus coat protein. The incorporation of a non-native adenoviral coat protein into an adenovirus and additional manipulations of the adenoviral coat protein are described in, for example, U.S. Pat. Nos. 5,559,099; 5,712,136; 5,731,190; 5,770,442; 5,846,782; 5,962,311; 5,965,541; and 6,057,155; and International Patent Applications WO 96/07734, WO 96/26281, WO 97/20051, WO 98/07865, WO 98/07877, WO 98/40509, WO 98/54346, and WO 00/15823. Construction of viral vectors, in particular adenoviral vectors, is described in, for example, International Patent Applications WO 98/56937 and WO 99/15686. Thus, the invention preferably provides an isolated and purified nucleic acid (and conservatively modified variants thereof) encoding a recombinant adenovirus coat protein.

A “conservatively modified variant” is a variation on the nucleic acid sequence that results in a conservative amino acid substitution. A “conservative amino acid substitution” is an amino acid substituted by an alternative amino acid of similar charge density, hydrophilicity/hydrophobicity, size, and/or configuration (e.g., Val for Ile). In comparison, a “nonconservatively modified variant” is a variation on the nucleic acid sequence that results in a nonconservative amino acid substitution. A “nonconservative amino acid substitution” is an amino acid substituted by an alternative amino acid of differing charge density, hydrophilicity/hydrophobicity, size, and/or configuration (e.g., Val for Phe). The means of making such modifications are well known in the art and also can be accomplished by means of commercially available kits and vectors (for example, those available from New England Biolabs, Inc., Beverly, Mass.; Clontech, Palo Alto, Calif.).

Furthermore, the present inventive adenoviral coat proteins, and their corresponding nucleic acid sequences, can be incorporated into an adenoviral vector, as described in, for example, U.S. Pat. Nos. 5,559,099; 5,712,136; 5,731,190; 5,770,442; 5,962,311; 5,965,541; 5,846,782; and 6,057,155; and International Patent Applications WO 96/26281, WO 98/07877, WO 98/07865, WO 98/40509, and WO 98/54346. Such a vector can be rendered replication-incompetent by deleting some of the genes required for viral replication (e.g., E1a, E1b, E2 and/or E4). Preferably, the adenoviral vector is deficient in one or more gene functions of any or all of the E1 (e.g., E1a and/or E1b), E2 (e.g., E2A), and/or E4 (e.g., ORF-6) regions. In addition, the adenoviral vector can be an amplicon wherein, for instance, only the 5′ and 3′ inverted terminal repeats (ITRs) and sequences required for viral packaging are present. Suitable replication incompetent adenoviral vectors are disclosed in International Patent Applications WO 95/34671 and WO 97/21826. The expendable E3 region can be deleted (in whole or in part) to allow additional room for a larger DNA insert, while retaining a replication competent adenoviral vector, if desired. Alternatively, the vector can be replication competent, or conditionally replication competent, to permit high copy-number of a transgene to be produced in the target cells and/or increase cell to cell spread among target cells. Furthermore, any of the above-described alterations of the adenoviral vector can be such that it does not productively infect packaging cells, for example, HEK-293 cells. The adenoviral vector can be conjugated to a non-native ligand, which is described in further detail herein.

The adenoviral vector can comprise one or more chimeric adenoviral coat proteins, such as the inventive coat proteins or other types of chimeric coat proteins. For example, a chimeric adenovirus coat protein can comprise a non-native amino acid sequence, wherein the chimeric adenovirus coat protein directs entry into a cell of an adenoviral vector comprising the chimeric adenovirus coat protein that is more efficient than entry into a cell of an adenoviral vector that is identical except for comprising a wild-type adenovirus coat protein rather than the chimeric adenovirus coat protein. Another type of chimeric adenovirus coat protein can comprise a non-native amino acid sequence that serves to increase efficiency by decreasing non-target cell transduction by the adenoviral vector. Also, the non-native amino acid sequence can serve to increase efficiency by decreasing recognition of the adenoviral vector by the immune system.

The non-native amino acid sequence of the chimeric adenovirus coat protein generally comprises a deletion of native amino acids, a substitution of non-native amino acids for native amino acids, or an insertion of non-native amino acids. The amino acid alterations can comprise from about 1 to about 750 amino acids, preferably from about 1 to about 500 amino acids, and optimally from about 1 to about 300 amino acids. It also is desirable that the altered region comprises a smaller region that encodes less than about 200 amino acids, preferably less than about 100 amino acids, and optimally less than about 50 amino acids. The non-native nucleic acid sequence can be inserted into or in place of an internal coat protein sequence, or, alternatively, the non-native amino acid sequence can be at or near the C-terminus or N-terminus of the chimeric adenovirus coat protein. In addition, the non-native amino acid sequence can be linked to the chimeric adenovirus coat protein by a spacer sequence of from about 3 amino acids to about 30 amino acids.

A chimeric coat protein can, for example, comprise a non-native amino acid sequence that allows an adenoviral vector incorporating such a protein to elicit less RES clearance in a host animal than a corresponding wild-type adenovirus. To facilitate this, the coat protein can be engineered to lack a native glycosylation or phosphorylation site or contain a peptide that binds an agent that masks the vector from recognition by neutralizing antibodies or the RES or itself masks the vector. Suitable agents include, for instance, polyethylene glycol (PEG), peptides that bind serum components, and the like. Alternatively, the coat protein can be engineered to contain non-native residues that facilitate post-translational modification (e.g., one or more non-native cysteine residues can facilitate post-translational conjugation by way of disulfide bonding). The vector also can be functionally linked (e.g., conjugated) to a lipid derivative of polyethylene glycol comprising a primary amine group, an epoxy group, or a diacylglycerol group. Without being bound by any particular theory, such modifications are believed to mask the adenoviral vector, at least in part, from scavenging by the cells of the reticulo-endothelial system (RES). Indeed, the chimeric coat protein is preferably linked to a molecule that masks the vector from recognition by the RES and neutralizing antibodies in order to increase half-life of the vector in the bloodstream.

In addition, an adenoviral vector of the present invention can include one or more non-native nucleic acids for transcription. A non-native nucleic acid can be any suitable nucleic acid sequence (e.g., gene), and desirably is either a therapeutic gene (i.e., a nucleic acid sequence encoding a product that effects a biological, preferably a therapeutic, response either at the cellular level or systemically), or a reporter gene (i.e., a nucleic acid sequence which encodes a product that, in some fashion, can be detected in a cell). Preferably a non-native nucleic acid is capable of being expressed in a cell into which the vector has been internalized. Preferably the non-native nucleic acid exerts its effect at the level of RNA or protein. For instance, a protein encoded by a transferred therapeutic gene can be employed in the treatment of an inherited disease, such as, e.g., the cystic fibrosis transmembrane conductance regulator cDNA for the treatment of cystic fibrosis. Alternatively, the protein encoded by the therapeutic gene can exert its therapeutic effect by effecting cell death. For instance, expression of the gene in itself can lead to cell killing, as with expression of the diphtheria toxin. Alternatively, a gene, or the expression of the gene, can render cells selectively sensitive to the killing action of certain drugs, e.g., expression of the HSV thymidine kinase gene renders cells sensitive to antiviral or other toxic compounds including aciclovir, ganciclovir, and FIAU (1-(2-deoxy-2-fluoro-β-D-arabinofuranosil)-5-iodouracil). Moreover, the therapeutic gene can exert its effect at the level of RNA, for instance, by encoding an antisense message or ribozyme, a protein which affects splicing or 3′ processing (e.g., polyadenylation), or a protein affecting the level of expression of another gene within the cell (i.e., where gene expression is broadly considered to include all steps from initiation of transcription through production of a processed protein), perhaps, among other things, by mediating an altered rate of mRNA accumulation, an alteration of mRNA transport, and/or a change in post-transcriptional regulation. Exemplary genes for inclusion into adenoviral vectors include, for instance, an angiogenic gene (e.g., a VEGF), an anti-angiogenic gene (e.g. PEDF), a cytokine, a vasodilator, a transcription factor, a neurotrophic factor (e.g., CNTF), an atonal-associated peptide (e.g., HATH1), and the like. The sequences of many desirable native nucleic acids are known in the art.

To facilitate expression, the non-native nucleic acid can be operably linked to an adenoviral or a non-adenoviral promoter. Any such promoter can be employed, such as a constitutive promoter (e.g., a viral immediate early promoter), a tissue-specific promoter, a regulatable promoter (e.g., metallothionin promoter, tetracycline-responsive promoter, RU486-responsive promoter, etc.), or other desired promoter. The non-native nucleic acid can be inserted into any suitable region of the adenoviral vector. For example, where the vector is replication incompetent, the DNA segment can be inserted into an essential viral genome location (e.g., the E1 region).

The inventive adenoviral vectors can be used to infect cells. Accordingly, the invention provides a method of infecting a cell by contacting a cell with an adenoviral vector as described above. As the adenoviral vector has a non-native ligand, the adenoviral vector can be targeted to infect the cell in accordance with the inventive method. Typically, the non-native nucleic acid encodes a protein as discussed above. In such instance, the method permits the nucleic acid to be expressed within the cells to produce the protein. Accordingly, the inventive adenoviral vectors and methods can be used in gene transfer applications, such as are commonly employed in research and, increasingly, clinical applications. For delivery into a host animal, an adenoviral vector of the present invention can be incorporated into a suitable carrier. As such, the present invention provides a composition comprising an adenoviral vector of the present invention and a pharmacologically acceptable carrier (e.g., a pharmaceutically- or physiologically-acceptable carrier). Any suitable preparation is within the scope of the invention. The exact formulation depends on the nature of the desired application (e.g., cell type, mode of administration, etc.), and many suitable preparations are set forth in U.S. Pat. No. 5,559,099.

The inventive vectors can be engineered by standard methods of vector construction. For example, a gene encoding the coat protein can be introduced into a packaging cell along with the rest of the adenoviral genome. The gene encoding the coat protein, thus, can be recombined into the adenoviral genome (typically in place of the wild-type counterpart gene) or it can be introduced on a separate nucleic acid molecule (e.g., a plasmid), from which it is expressed during viral replication. The coat protein then will associate with the adenoviral capsid in a similar manner as its wild-type counterpart. Thereafter, the inventive vector can be isolated and purified by standard techniques. Methods of viral vector construction and purification are described in, for example, U.S. Pat. No. 6,168,941 and International Patent Applications WO 98/56937, WO 99/15686, and WO 99/54441.

Pseudo-Receptor System

Often, viral vectors do not readily infect their native host cell via the native receptor because the viral vector's ability to bind receptors is significantly attenuated (through, for example, incorporation of a chimeric adenoviral coat protein, such as the type discussed herein).

The present invention provides a system, which includes a cell expressing a non-native cell-surface receptor (a pseudo-receptor) and a virus having a ligand for that receptor. Preferably, the cell is in vivo. Also provided is a system wherein a transgenic animal comprises the cell expressing a non-native cell-surface receptor and a virus having a ligand for that receptor. In this system, the cell having a non-native cell-surface receptor can be localized within specific tissue of the transgenic animal through, for example, tissue-specific regulation of the receptor. Tissue-specific regulation of a receptor can be achieved by operably linking the coding sequence of the non-native cell-surface receptor to a tissue-specific promoter.

The cell having a non-native cell-surface receptor can be in any suitable environment, such as alone, in a population of cells comprising the cell (or cells) having a non-native cell-surface receptor and cells not having the non-native cell-surface receptor (e.g., a mixture of cells), and localized within specific tissue of an animal (e.g., a transgenic animal) through, for example, tissue-specific regulation of the receptor. Thus, the cell can be an individual cell. Alternatively, the cell can be part of a collection of cells in vitro or in vivo. For example, the cell can be part of a cell culture maintained using standard techniques. The cell can additionally be present in a population of cells such as a tissue, an organ, an organ system, or an organism, such as a plant or an animal. The animal can be a mammal, e.g., a test or laboratory animal (mammal) such as a mouse, rat, monkey, pig, or goat, although the animal also can be human.

If an animal comprises the cell having a non-native cell-surface receptor, the animal can comprise alterations other than the expression of a non-native cell-surface receptor. For example, the animal can be a knock-out model wherein the animal is deficient in at least one gene function. Alternatively, the genome of the animal can be manipulated such that the animal displays a diseased phenotype. Desirably, a transgenic animal comprises the cell having a non-native cell-surface receptor. Transgenic animals are extremely useful tools for genetic, physiological, and pharmacological research. “Transgenic” is a term understood in the art and refers to the introduction of a foreign nucleic acid sequence into an organism's genome. Methods of generating transgenic animals are well understood in the art and include, for example, microinjection of foreign DNA into a fertilized ovum, which then is allowed to mature into the animal. However, the animal need not be “transgenic” and can comprise a foreign nucleic acid in, for example, episomal form.

The cell can be of any suitable type capable of being transduced by the viral vector. The cell comprising the non-native cell-surface receptor of the system can be produced by any suitable method. For example, a DNA (e.g., an oligonucleotide, plasmid, cosmid, viral, or other vector) containing a nucleic acid encoding the non-native receptor can be introduced into a cell by any suitable means. Suitable methods of introducing DNA into a host cell include, for instance, electroporation, precipitation and co-incubation of the vector with suitable salts (e.g., CaCl₂ or LiCl), particle bombardment, needle-mediated direct injection, transduction, infection (e.g., mediated by a viral coat-protein), as well as other suitable methods described in, for example, Sambrook et al., supra, and Ausubel et al., Current Protocols in Molecular Biology, Greene Publishing Associates and John Wiley & Sons, New York, N.Y. (1994). Preferably, the DNA, desirably present in a vector, also encodes an agent permitting the cells harboring it to be selected (e.g., the vector can encode resistance to antibiotics which kill cells not harboring the vector). The DNA also can encode a marker peptide, such as green fluorescence protein, for easy identification of transduced cells. In some embodiments, the vector will recombine with the cell genome to produce a transduced cell expressing the non-native receptor. In other embodiments the DNA will remain in episomal form, thereby allowing transient expression of non-native cell-surface receptors.

Therefore, when the cell is within or on an animal, the cell can be transfected with the nucleic acid encoding the non-native receptor. Alternatively, the cell can be within a transgenic animal, the genome of which harbors the receptor nucleic acid. In several embodiments, it is desirable to engineer the animal such that the nucleic acid encoding the non-native cell-surface receptor is under the control of a promoter active in discrete tissues or organ types, i.e., tissue-specific promoters. Tissue-specific promoters for expression of the non-native cell-surface receptor are available for most tissues and allow for the nucleic acid to target areas or cells of interest. Similarly, production of a non-native cell-surface receptor can be under the control of other types of inducible promoters, such as hypoxia-driven promoters, temperature-sensitive promoters, and the like, which permit assessment of the function of a gene product under particular environmental conditions in vitro and in vivo.

With respect to the non-native cell-surface receptor, by “non-native” is meant any receptor that is not naturally found in a host cell. In an organism, the non-native receptor desirably is not native to the organism. Alternatively, the non-native receptor can be found in a cell type or tissue of the organism, but is not naturally present on the host cell. For example, a receptor commonly found on hepatocytes but not on cardiac cells is non-native to cardiac cells. In addition, a non-native receptor can be a receptor that is introduced into a host cell that has been previously manipulated to ablate the receptor (i.e., a knock-in). For example, the non-native cell-surface receptor can be CAR that is expressed in a tissue-specific manner in the desired host cells subsequent to knocking out the receptor in the animal. Preferably, the non-native receptor mediates more efficient cellular transduction in cells of the system than cells that do not comprise the non-native cell-surface receptor. For example, transduction of cells comprising the non-native cell-surface receptor is at least about 10-fold greater than transduction of cells without the non-native receptor. Preferably, infection of the cells of the system is at least about 50-fold greater, more preferably at least about 100-fold greater, than cells not expressing the non-native receptor. Internalization of the gene transfer vector into (e.g., transduction or viral infection of) cells other than those of the system need not be completely abolished, although it desirably is substantially or entirely avoided. Native binding of the gene transfer vector can be ablated to more efficiently target delivery of the gene transfer vector to desired cells comprising the non-native cell-surface receptor.

In addition, the receptor present on the cells of the system can be a receptor that is naturally present within or on the host cells (i.e., the receptor is native to the host cells), but is overexpressed compared to wild-type cells. Overexpression of the receptor in the cell provides for a greater number of binding sites for the ligand of the gene transfer vector, and, therefore, the gene transfer vector will bind and enter the cells of the system with greater efficiency than other cells. Overexpression can be achieved by introducing many nucleic acid molecules encoding the receptor into the cell to provide more substrate for transcription reactions. Additionally, the nucleic acid sequence encoding the receptor can be operably linked to a stronger promoter than that naturally driving expression of the nucleic acid sequence. A suitable promoter also can be selected such that temporal control of the expression of the receptor can be achieved.

If viral propagation is required, any cell capable of supporting viral growth is a suitable cell for use in the present inventive system. If the virus lacks genes essential for viral replication, preferably the cell expresses complementing levels of such gene products (see, e.g., International Patent Application WO 95/34671 and U.S. Pat. Nos. 5,658,724 and 5,804,413). When the virus is an adenoviral vector, preferably the cell line of the present invention is derived from HEK-293 cells, such as 293-ORF6 cells. Also preferably, the cell line is derived from lung carcinoma cells (e.g., non-small cell lung carcinoma cells), renal carcinoma cells, human retinal cells, human embryonic retinal (HER) cells, HeLa cells, CHO cells, 786-0 cells, G-402 cells, ARPE-19 cells, KB cells, and Vero cells. Suitable lung carcinoma cells include, for example, the cell lines NC1-H2126 (American Type Culture Collection (ATCC) No. CCL-256), NCI-H23 (ATCC No. CRL-5800), NCI-H1299 (ATCC No. CRL-5803), NCI-H322 (ATCC No. CRL-5806), NCI-H358 (ATCC No. CRL-5807), NCI-H810 (ATCC No. CRL-5816), NCI-H1155 (ATCC No. CRL-5818), NCI-H647 (ATCC No. CRL-5834), NCI-H650 (ATCC No. CRL-5835), NCI-H1385 (ATCC No. CRL-5867), NCI-H1770 (ATCC No. CRL-5893), NCI-H1915 (ATCC No. CRL-5904), NCI-H460 (ATCC No. HTB-177), NCI-H520 (HTB-182), and NCI-H596 (ATCC No. HTB-178), the squamous/epidermoid carcinoma lines Calu-1 (ATCC No. HTB-54), HLF-a (ATCC No. CCL-199), NCI-H292 (ATCC No. CRL-1848), NCI-H226 (ATCC No. CRL-5826), Hs 284.Pe (ATCC No. CRL-7228), SK-MES-1 (ATCC No. HTB-58), and SW-900 (ATCC No. HTB-59), the large cell carcinoma line NC1-H661 (ATCC No. HTB-183), and the alveolar cell carcinoma line SW-1573 (ATCC No. CRL-2170). When the virus is a herpesvirus, preferably the cell line of the present invention is derived from VERO cells. Preferably, the system can support viral growth for at least about 10 passages (e.g., about 15 passages), and more preferably for at least about 20 passages (e.g., about 25 passages), or even 30 or more passages. The non-native cell-surface binding site is a substrate molecule, to which a viral vector having a ligand (e.g., a non-native ligand) selectively binding that substrate can bind the cell and thereby promote cell entry. The binding site can recognize a non-native ligand incorporated into the adenoviral coat or a ligand native to a virus. For example, when the non-native viral ligand is a tag peptide such as hemagluttenin, the binding site can be an immunoglobulin molecule or derivative thereof, such as a single chain antibody (ScAb) receptor recognizing the tag. Alternatively, the receptor can recognize an epitope present in a region of a mutated fiber knob (if present), or even an epitope present on a native adenoviral coat protein (e.g., on the fiber, penton, hexon, etc.). Alternatively, if the non-native ligand recognizes a cell-surface substrate (e.g., membrane-bound protein), the binding site can comprise that substrate. The cell line can express a mutant receptor with decreased ability to interact with the cellular signal transduction pathway (e.g., a truncated receptor, such as NMDA (Li et al., Nat. Biotech., 14, 989 (1996))) or attenuated ability to act as an ion channel, or other modification. Infection via such modified proteins minimizes the secondary effects of viral infection on host-cell metabolism by reducing the activation of intracellular messaging pathways and their various response elements. The choice of binding site depends to a large extent on the nature of the viral vector. However, to promote specificity of the virus for a particular cell type, the binding site preferably is not a native mammalian receptor for the appropriate wild-type virus. Thus, for example, when the gene transfer vector of the system is an adenovirus, the non-native cell-surface receptor is preferably a non-adenoviral receptor, which desirably binds a substrate other than a native (i.e., wild-type) adenoviral ligand. In some instances, it may be desirable for the cell-surface receptor to be native to the cell, but bind a non-native adenoviral ligand (i.e., a ligand not present on wild-type adenovirus).

The binding site of the receptor must be expressed on the surface of the cell to be accessible to the virus. Hence, where the binding site is a protein, it preferably has a leader sequence and a membrane-tethering domain to promote proper integration into the membrane (see, e.g., Davitz et al., J. Exp. Med. 163, 1150 (1986)). Moreover, to better facilitate viral entry into the cell, the receptor can comprise a cytoplasmic portion able to mediate viral internalization. Thus, for example, the receptor protein can comprise a transmembrane domain fused to an internalization domain derived from an integrin (e.g., α5 integrin or αvβ5) cytoplasmic domain or LDL cytoplasmic domain. This internalizing domain can be any suitable domain that increases the number of adenoviral vectors internalized when the receptor is present on the surface of a cell. Additionally, therefore, the invention provides a cell-surface receptor (e.g. a non-native, non-adenovirus cell-surface receptor) comprising a first domain and a second domain. The first domain of the present inventive cell-surface receptor binds a viral vector having one or more chimeric coat proteins, and the second domain facilitates internalization of the vector into a cell. Preferably, the second domain actively facilitates internalization of the vector into the cell. By “actively facilitates internalization” is meant that internalization is not achieved by diffusion or passive movement across the cell membrane such as occurs, for instance, by receptors comprising a transmembrane domain only. Second domains that actively facilitate internalization are frequently associated with an internalization domain associated with, for example, clatherin-coated pits, although such an association is not required for the present invention. By “non-adenovirus cell-surface receptor” is meant a cell-surface receptor that does not bind wild-type adenovirus. Alternatively, the second domain can comprise a chemical linkage to the cell membrane, such as a glycerol-phosphate-inositol (GPI) linkage, which also allows internalization of the virus. The present invention further provides a cell expressing the inventive cell-surface receptor.

The present inventive system also can be used to propagate a virus by infecting a cell of the inventive system with a virus. The cell is then maintained such that viral particles are produced in the cell. Finally, the virus produced within the cell can then be recovered by standard methods. Using this method, viral vectors that no longer express a binding site for native receptor can be grown in sufficient titers. In this regard, one of ordinary skill in the art will appreciate that a cell comprising a non-adenoviral cell-surface receptor, which can be native to the cell, also can be used to propagate an adenoviral vector having a non-native ligand that binds the non-adenoviral cell surface receptor. The native-receptor binding capabilities of the adenoviral vector can be ablated, if desired, using the methods described herein. Alternatively, the present inventive system can be used to assay for gene function. After infecting a cell of the system with a virus, the cell can be maintained and physiological alteration assayed. Where the cell is within a transgenic animal, the invention provides a convenient, simple method to investigate a gene's functions in vivo. For example, an adenoviral vector comprising a lung-specific gene to be assayed can be administered to a transgenic animal having a cell of the present inventive system localized to lung tissue (e.g., under control of a lung-specific promoter). The effects of adenoviral expression of the lung-specific gene then can be determined.

Assaying Gene Function and Identifying Nucleic Acid Sequences

The present inventive system represents a significant advancement with respect to tools used in virology, genomics, and pharmacology research. For instance, the present inventive system can be used to assay for gene function. A cell having a non-native cell surface receptor (such as those cells described herein) is infected with a gene transfer vector comprising a ligand that binds the non-native cell surface receptor of the cell. Preferably, the gene transfer vector is a viral vector, as herein described. Also preferably, the ligand is a non-native ligand. The cell is maintained and assayed for physiological alterations. Changes in cell physiology can be determined using a variety of methods, such as those known in the art. Changes in cellular physiology can include, for example, increased cell proliferation, cell transformation, alterations in gene expression, alterations in cellular mobility, cell death, changes to the cell cycle, increased sensitivity or resistance to toxins, metabolic inconsistencies, alterations in protein-protein interactions, the ability to regulate an enzyme or an ion channel, and the like. Where the cell is within an animal, e.g., a transgenic animal, the invention provides a convenient method to investigate a gene's functions in vivo. Adenoviral vectors are preferred in functional genomics in that minimal perturbation of target cells occurs, and adenoviral vectors have been demonstrated to efficiently transduce cells in vivo. However, although adenovirus is preferred, other gene transfer vectors can be used in assaying for gene function or identifying nucleic acid sequences of interest including, but not limited to, retroviral vectors, parvoviral vectors (e.g., adeno-associated virus), herpes virus, lentiviral vectors, and plasmids. In this regard, methods of conjugating a gene transfer vectors to a non-native ligand is understood in the art and well within the skill of the ordinary artisan.

The present inventive system also can be used to screen genetic libraries to isolate a nucleic acid encoding a product comprising a desired property. The present inventive method of isolating a nucleic acid of interest comprises infecting the cells of the system with a library of gene transfer vectors (i.e., viruses of the present inventive system) wherein each member of the library of gene transfer vectors comprises a nucleic acid encoding a product comprising a potentially desired property. The cells comprising the library are assayed for a desired property, and the member of the library of gene transfer vectors comprising the nucleic acid encoding the product comprising the desired property is isolated. The nucleic acid of interest can be isolated from the gene transfer vector (i.e., virus) and, if desired, sequenced. Preferably, an animal comprises the cells that are infected by the library of gene transfer vectors.

A library of gene transfer vectors (e.g., a library of viral vectors) preferably comprises or consists of a multiplicity of vectors, preferably viral vectors comprising a multiplicity of genetic elements. Any number of individual gene transfer vectors can make up the library of gene transfer vectors. Similarly, the complexity of the library of gene transfer vectors can vary according to the particular embodiment. By “complexity” is meant the number of unique individuals in the library. The complexity of a library can be 1. The complexity of the library of gene transfer vectors can be about 1 to about 10¹¹ unique individuals (i.e., 10, 50, 100, 500, 1000, 5000 or more unique individuals). Preferably, the complexity of the library of gene transfer vectors is about 1 to about 10⁶ unique individuals, although libraries of higher complexity (i.e., 10⁷, 10⁸, 10⁹, or 10¹⁰ unique individuals) are suitable for use in the present inventive method.

Each member of the library of gene transfer vectors comprises a nucleic acid encoding a product comprising a potentially desired property. The nucleic acids can be obtained from any source and in any manner. For example, the nucleic acids can be genomic DNA obtained from a source that has not been genetically modified or has been modified to exhibit a particular phenotype. The nucleic acids can comprise cDNA or can be synthetically made using routine methods known in the art. The nucleic acids can comprise pieces of larger molecules of DNA fragmented by chemical, enzymatic, or mechanical means. The nucleic acids of the library also can comprise polymerase chain reaction (PCR) products of DNA segments, and the like. Preferably, the nucleic acids are obtained from a population of DNA comprising a multiplicity of genetic elements. The probability of identifying and isolating a nucleic acid of interest depends greatly on the diversity of the genetic library. It is, therefore, advantageous to mutate the nucleic acids to obtain optimal diversity in the library of gene transfer vectors. However, mutation of the nucleic acids is not required and, in some embodiments, not desired.

Within members of the library, the nucleic acid can be the same, i.e., the nucleic acids encode the same product or variations thereof, or different, i.e., the nucleic acids encode different products. “Product” is meant to include, for instance, a peptide or functional nucleic acid sequence. As used herein, “peptide” refers to an amino acid sequence of any length. Therefore, “peptide” is meant to encompass peptides, polypeptides, proteins, and fragments thereof. By “functional nucleic acid sequence” is meant a nucleic acid sequence, i.e., DNA or RNA, that performs a function or has an activity within a cell. An example of a functional nucleic acid is antisense RNA that impedes transcription or translation of a DNA or RNA sequence. Functional nucleic acid sequences also include, but are not limited to, promoters, enhancers, enzyme binding sites, splice sites, and ribozymes.

The skilled artisan will appreciate the utility of the present inventive system in screening genomic libraries in vivo and screening for therapeutic factors. Most previously described methods of screening nucleic acid sequences comprise in vitro expression of encoded gene products. The expressed gene product is either identified in vitro or administered as a peptide in vivo. The library of gene transfer vectors, e.g., adenoviruses, described herein allows for efficient and, optionally, selective delivery of a nucleic acid to cells to produce a product comprising a potentially desirable property in vivo, wherein the gene product is expressed and screened for function. Methods of screening libraries are discussed, for example, in U.S. patent application Ser. No. 09/780,526.

Accordingly, gene function can be observed in specific tissues or under specific conditions through use of the present inventive system. For example, an adenoviral vector comprising a lung-specific gene to be assayed can be administered to an animal having the cells of the present inventive system localized to lung tissue (e.g., production of the non-native cell-surface receptor in the cell is under the control of a lung-specific promoter). The effects of adenoviral expression of the lung-specific gene then can be determined, which permits assessment of the effects of the desired transgene within predefined tissues. By targeting gene transfer to achieve pre-defined tissue-specific peptide production, as opposed to controlling peptide production with a tissue-specific promoter, background and other non-specific activities of the gene transfer vector in other cells is minimized.

The in vivo model for screening the library of gene transfer vectors, e.g., adenoviruses, depends on the desired property encoded by the DNA fragments. In one aspect, the animal is healthy, and the cells are assayed to detect any change in phenotype in a wild-type animal. Alternatively, the animal comprising the cells is afflicted with a disease in order to select a nucleic acid encoding, for instance, a therapeutic factor. By “therapeutic factor” is meant a peptide or functional nucleic acid sequence that alleviates or inhibits, in whole or in part, a disease or ailment. As used herein, a therapeutic factor can affect, for example, the nervous system, genitourinary ailments, cancer, infectious disease, and cardiovascular abnormalities, as well as other health nuisances. Therapeutic factors identified by the present inventive method can be used to treat, for example, sleep disorders, ALS (Lou Gehrig's Disease), Alzheimer's Disease, epilepsy, multiple sclerosis, Parkinson's Disease, peripheral neuropathies, Schizophrenia, depression, anxiety, spinal cord injury, traumatic brain injury, or acute, chronic, or inflammatory pain. Therapeutic factors can be identified to treat genitourinary ailments, which include, for example, benign prostatic hyperplasia (BPH), impotence, neurogenic bladder, urinary incontinence, kidney failure, and end stage renal disease. Therapeutic factors useful in treating cancer, such as, for example, cancer of the bladder, brain, breast, colorectal, esophageal, head and neck, liver/hepatoma, lung, melanoma, ovarian, pancreatic, prostate, stomach, testicular, uterine/endometrial, leukemias, and lymphomas, also can be identified using the present inventive method. Therapeutic factors can be identified to treat infectious diseases that include, but are not limited to, chlamydia, herpes, malaria, human papilloma virus (HPV), AIDS/HIV, pneumococcal pneumonia, influenza, meningitis, hepatitis, and tuberculosis. Therapeutic factors for treating cardiovascular diseases, such as, for example, neovascular diseases, ischemia, congestive heart failure, coronary artery disease, arrhythmia, athlerosclerosis, increased LDL/HDL ratios, restenosis after angioplasty or in-stent restenosis, stroke, sickle cell anemia, and hemophilia, can be identified, as well as therapeutic factors associated with the alleviation of, for example, obesity, organ transplantation/transplant rejection, osteoporosis, alopecia, arthritis, allergies (such as to ragweed, pollen, and animal dander), cystic fibrosis, diabetes, macular degeneration, glaucoma, and hearing loss.

Animal models of a number of diseases and disorders, including those disease states identified above, are available commercially for use in the present inventive method. For additional information regarding animal models of disease, see, for example, Immunodeficient Mice in Oncology (Contributions to Oncology, Vol. 42), Fiebig & Berger (Editors), S. Karger Publishing (July 1992); Man and Mouse: Animals in Medical Research, William D. M. Paton, ASIN: 0192861468; Genetic Models of Immune and Inflammatory Diseases (Serono Symposia Usa), Abbas & Flavell, Eds., USA Serono Symposia, ASIN: 0387946497; Urinary System (Monographs on Pathology of Laboratory Animals), 2^{nd} Ed., Jones et al. (Editors), Springer Verlag (June 1998), ISBN: 0944398766; What's Wrong with My Mouse?: Behavioral Phenotyping of Transgenic and Knockout Mice, Jacqueline N. Crawley, John Wiley & Sons (Mar. 10, 2000), ISBN: 0471316393; The Scid Mouse: Characterization and Potential Uses (Current Topics in Microbiology and Immunology, Vol. 152), R. W. Compans (Editor), Springer Verlag (May 1990), ISBN: 0387515127; Strategies in Transgenic Animal Science, Monastersky & Robi (Editors), Amer. Society for Microbiology (July 1995), ISBN: 1555810969; Pathology of Tumours in Laboratory Animals: Tumours of the Mouse, 2^{nd} Ed., Turusov & Mohr (Editors), Iarc Scientific Publications, Vol. 002, No. 111, Oxford Univ. Press (February 1994), ISBN: 9283221117; Laboratory Animals in Vaccine Production and Control: Replacement, Reduction, and Refinement (Developments in Hematology and Immunology), Hendriksen & Nijhoff (October 1988), ISBN: 0898383986; Motor Activity and Movement Disorders: Research Issues and Applications (Contemporary Neuroscience), Sanberg et al. (Editors), Humana Pr. (January 1996), ISBN: 0896033279; Cardiovascular and Musculoskeletal Systems (Monographs on Pathology of Laboratory Animals), Jones et al. (Editors), Springer Verlag (September 1991), ISBN: 0387538763; CRC Handbook of Animal Models for the Rheumatic Diseases, Greenwald & Diamond (Editors), CRC Press (November 1988), ISBN: 0849329884; Experimental and Genetic Rat Models of Chronic Renal Failure, Gretz & Strauch, S. Karger Publishing (February 1993), ISBN: 3805554990; Central Nervous System Diseases: Innovative Animal Models from Lab to Clinic (Contemporary Neuroscience), Emerich et al. (Editors), Humana Pr. (November 1999), ISBN: 089603724X; Experimental Models of Diabetes, John H. McNeill (Editor), CRC Press (January 1999), ISBN: 0849316677; Laboratory Animals: An Introduction for Experimenters, 2^{nd} Ed., A. A. Tuffery (Editor), John Wiley & Son Ltd. (Jun. 27, 1995), ISBN: 0471952575; Animal Models in Cardiovascular Research (Developments in Cardiovascular Medicine, Vol. 153), David R. Gross, Kluwer Academic Publishers (June 1994), ISBN: 0792327128; Anxiety, Depression, and Mania (Animal Models of Psychiatric Disorders, Vol. 3), Soubrie (Editor), S. Karger Publishing (December 1990), ISBN: 3805552475; Toxicity Assessment Alternatives: Methods, Issues, Opportunities, Salem & Katz (Editors), Humana Pr (July 1999), ISBN: 0896037878; and Pathobiology of the Aging Mouse: Nervous System, Special Senses (Eye and Ear), Digestive System, Integumentary System and Mammary Gland, and Musculos, Mohr et al. (Editors), Int'l Life Sciences Inst., Vol. 2 (October 1996), ISBN: 0944398464.

With respect to therapeutic factors, the gene transfer vector (i.e., adenoviral vector) and, subsequently, the nucleic acid encoding the product with therapeutic properties, is selected by observing a change in the disease state of the animal. For example, when the desired therapeutic factor is an angiogenic peptide, neovascularization is detected in the animal to isolate the nucleic acid of interest. If desired, multiple rounds of screening can be performed to isolate a nucleic acid encoding the most effective therapeutic factors. Indeed, the present inventive method can be performed at least 2, 3, 4, or more times (e.g., at least 5, 7, 10, 15, or 20 or more times) in order to isolate a nucleic acid of interest.

In some embodiments, it may be useful to examine the function of an unknown factor in the presence of known gene products in a particular tissue or cell type. For example, previously described genomic libraries have been utilized to identify key genes involved in specific diseases or biological functions. However, once a gene is identified with a central role in a function or disease, it is not obvious how functionally related genes or genes in the same cellular pathway can be identified. Identifying genes that might be structurally and even functionally unrelated, but could synergize or repress each other in the context of a multi-factorial biological function or disease, is not trivial. In view of the need in the art to identify structurally- or functionally-related gene products and related gene functions centered around a known function, the present invention further provides a method of identifying functionally-related coding sequences using the present inventive system. The method comprises infecting the cells of the system with the library of gene transfer vectors of the system, wherein each member of the library comprises a first heterologous DNA encoding a first gene product and a second heterologous DNA encoding a second gene product. The first DNA is common to each member of the library of vectors. In other words, a gene or a set of genes is kept constant in all individual members of the library. The second DNA varies between the members of the library of gene transfer vectors (e.g., the library of viral vectors). The method further comprises comparing the activity of the gene products encoded by the library of gene transfer vectors with the activity of the first gene product encoded by a vectorcomprising the first heterologous DNA but not comprising the second heterologous DNA. Preferably, the gene transfer vector is a virus, such as an adenovirus.

The multi-gene vector library and methods of use can be employed to identify gene products which affect some activity, such as which enhance or repress the activity of the known gene product. For example, the second DNA can encode a putative neurotrophic factor while the common gene product encoded by the first DNA is a neurotrophic factor. To identify functionally related coding sequences, the cells of the system are infected with the library of gene transfer vectors, and enhanced neuron survival is detected and compared to neuron survival provided by expression of the gene product alone. Contrariwise, the above-described method also can be utilized to identify factors that repress, not enhance, activity of the gene product. In some situations, it is advantageous to identify factors that do not affect the activity of a given gene product. The present invention thus allows for the identification of related gene functions centered around a known function, that of the first DNA-encoded gene product. Adenoviral vectors are particularly suited for use in such a library due to the available large insert capacity.

A library of gene transfer vectors (e.g., viral vectors), including a multi-gene library of gene transfer vectors, can be constructed using standard methods. By “heterologous” is meant that the DNA is non-native to the vector or is native to the vector, but is not located in its native location or position with the vector. One of ordinary skill in the art will appreciate that more than one “second” DNA can be inserted into each member of the library of gene transfer vectors. The first DNA and second DNA of the multi-gene gene transfer vector library preferably are operably linked to regulatory sequences necessary for expression of the encoded gene products. For example, preferably, the first DNA and/or the second DNA are operably linked to an inducible promoter. Also preferably, the first heterologous DNA and the second heterologous DNA are under the control of separate regulatory elements. Alternatively, the first heterologous DNA and the second heterologous DNA can be under the control of a bi-directional promoter. Manipulation of the heterologous DNAs can provide a means to detect interaction between the encoded gene products or to purify encoded gene products. For example, the first and/or second gene product can be fused to an antibody tag at the N- or C-terminus. Fusion of an antibody tag to the first and/or second gene product allows for the detection of physical interaction between proteins by the use of immunoprecipitation and mass spectrometry (see, for example, International Patent Application No. PCT/U.S.00/22234).

The first gene product preferably is known by the investigator, such that meaningful determination of the interaction between the first and second gene products can be accomplished. One of ordinary skill in the art will appreciate that the first gene product used will depend on the particular embodiment of the present invention. The first gene product can be, for example, an angiogenic factor, an anti-angiogenic factor, a transcription factor, a growth factor, a cytokine, an apoptotic agent, an anti-apoptotic agent, or a neurotrophic factor. For example, if the first gene product is associated with angiogenesis, the first gene product can be an endothelial mitogen, a factor associated with endothelial cell migration, a factor associated with vessel wall maturation, a factor associated with vessel wall dilation, and a factor associated with extracellular matrix degradation, and the like, such as a vascular endothelial growth factor (VEGF, e.g., VEGF₁₂₁). If the first gene product is associated with anti-angiogenesis, then the first gene product can be a pigment epithelial-derived factor (PEDF). As used herein, the first gene product, which is common to all the members of the gene transfer vector library, is preferably a factor that is required for a particular screening method to work. For example, when the goal of a screen is to identify factors that enhance angiogenesis in an animal, the first gene product preferably is an angiogenic factor, and the screen involves the detection of enhanced angiogenesis.

With respect to in vivo embodiments of the present invention, the gene transfer vector of the inventive system, e.g., the library of gene transfer vectors, desirably is administered to an animal in a physiologically acceptable (e.g., pharmaceutical) composition, which comprises the virus and a physiologically (e.g., pharmaceutically) acceptable carrier. Any suitable physiologically acceptable carrier (e.g., diluent) can be used within the context of the present invention. Appropriate formulations include, for example, aqueous and non-aqueous solutions, isotonic sterile injection solutions, which can contain anti-oxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. The formulations can be presented in unit-dose or multi-dose sealed containers, such as ampules and vials, and can be stored in a freeze-dried (lyophilized) condition requiring only the addition of the sterile liquid carrier, for example, water, immediately prior to use. Extemporaneous solutions and suspensions can be prepared from sterile powders, granules, and tablets of the kind previously described. Preferably, the physiologically acceptable carrier is a buffered saline solution.

The physiologically acceptable composition described herein can be delivered via various routes and to various sites in an animal body (see, e.g., Rosenfeld et al., Clin. Res., 39(2), 311A (1991)). One skilled in the art will recognize that although more than one route can be used for administration, a particular route can be more appropriate than another route, depending on the particular embodiment of the present inventive method. Local or systemic delivery can be accomplished by administration comprising application or instillation of the formulation into body cavities, inhalation or insufflation of an aerosol, or by parenteral introduction, comprising intramuscular, intravenous, peritoneal, subcutaneous, intraarterial, intraocular, and intradermal administration, as well as topical administration. Of course, the routes of administration discussed herein are merely exemplary. The present inventive methods are not dependent on the particular route of administration or dose of gene transfer vector administered.

Those of ordinary skill in the art can easily make a determination of the proper dosage of the virus to infect host cells, if desired. A variety of factors will impact the dosage that is administered to, for example, an animal. Specifically, the dosage will vary depending upon the particular method of administration and the particular gene transfer vector. If plasmids are used, about 0.5 ng to about 1000 μg can be administered. If using adenovirus, preferably, about 1×10⁴ to about 1×10¹⁵ adenovirus particles are administered to an animal, although more or less virus can be administered. Most preferably, about 1×10⁸ to about 1×10¹² adenovirus particles are administered to an animal. Wherein a library of gene transfer vectors is administered to an animal in order to select a nucleic acid encoding a gene product comprising a desired property, the amount of gene transfer vector administered should be sufficient to ensure efficient infection of, for example, target cells, and subsequent recovery of vectors. Any necessary variations in dosages and routes of administration can be determined by the ordinarily skilled artisan using routine techniques.

Controlled Gene Expression

Further provided by the invention is a method of controlled gene expression, which is accomplished by administering to an animal a selectively replication competent adenoviral vector, wherein the adenoviral vector comprises a first nucleic acid sequence and a targeting agent. Particularly, to accomplish controlled gene expression, the adenoviral vector further comprises a second non-native nucleic acid for selective replication. A targeting agent can be any suitable agent that directs binding of the adenoviral vector to a specific cell or cell type, such as a bi-specific molecule or a chimeric adenoviral coat protein, which are described previously and otherwise known in the art, or an agent for regulated gene expression, such as a tissue-specific promoter or a regulatable promoter.

Most of the adenoviral vectors used in research and therapeutic applications are replication-incompetent. That is, they do not productively infect cells. The present inventive method takes advantage of selective replication to control gene expression. Selective replication competence of the adenoviral vector can be achieved through modifications of the E1a and E1b regions of the adenoviral genome of the adenoviral vector. Selective replication can be used to amplify a given effect resulting from adenoviral vector administration, such that a much fewer number of adenoviral vectors need be administered to produce a similar effect. For example, the second non-native nucleic acid can be operably linked to an adenoviral or a non-adenoviral promoter, which can be regulatable, such as a tissue-specific promoter. Upon activation of this regulatable promoter, the second non-native nucleic acid is expressed, thus turning on specific genes in the adenoviral vector necessary for replication (e.g., genes of the E1 region). Alternatively, the adenoviral major late promoter can be used to activate replication. The first non-native nucleic acid need not be conditionally active upon expression of the second non-native nucleic acid. In this case, there will be a low level of expression if the adenoviral vector is not replicated. Upon replication, the levels of viral particles will increase, and thus the level of expression of the first non-native nucleic acid will also increase.

The first and second nucleic acids can be introduced into the viral genome and packaged into mature adenoviral virions by standard recombinant techniques. In this regard, any “backbone” adenoviral vector can be employed, which can be otherwise wild type or recombinant, depending on the desired qualities of the resulting adenoviral vectors. Thus, it should be appreciated that any of these selectively replication competent adenoviral vectors can include one or more chimeric adenoviral coat proteins, such as a hexon, penton base, fiber protein, etc., such as those described previously and otherwise known in the art. For example, the adenoviral vector can comprise a chimeric adenoviral coat protein unable to bind CAR and/or integrins on the cell surface. Indeed, it is preferable to employ such proteins to more tightly control adenoviral replication and gene expression. In this regard, preferably the first promoter is a tissue specific promoter, and the adenoviral vector is selectively targeted to the same tissue in which the first promoter is active.

Imaging Therapy

The invention also provides a method of therapy involving administration to an animal of an adenoviral vector having a first non-native nucleic acid, a second non-native nucleic acid, and a targeting agent. The first non-native nucleic acid specifically encodes a therapeutic agent, and the second non-native nucleic acid encodes an agent that facilitates imaging. A targeting agent can be any suitable agent that directs binding of the adenoviral vector to a specific cell or cell type, such as a bi-specific molecule or a chimeric adenoviral coat protein, which are described previously and otherwise known in the art, or an agent for regulated gene expression, such as a tissue-specific promoter or a regulatable promoter. It should be appreciated that any of the chimeric adenoviral coat proteins can be a hexon, penton base, or fiber protein, which are further described herein.

To facilitating imaging, the agent increases the ability to differentiate between targeted tissue and non-targeted tissue. Such targeted tissue includes, for example, inflamed tissue or the regions of a stroke. Imaging can be done by any suitable method. For example, the agent that facilitates imaging can any suitable such agent, e.g., a marker protein, such as luciferase, or a dye. Alternatively, the agent that facilitates imaging can be an enzyme that can concentrate radio-opaque or radioactive substances (e.g., heavy metals, iodine, etc.) into the cell. Any of these agents would serve to “mark” the bounds of the diseased tissue, e.g., the bounds of a tumor or lesion. They also, for example, would allow a clinician to determine if a tumor had metastasized. Metastasis is often used as a diagnostic marker, and this method would allow easy determination of metastases.

The present inventive method allows for administration of a therapeutic agent systemically to effect imaging of the discrete targeted tissue, e.g., for further therapeutic treatment. As such, it is not necessary to know the precise location of the tissue being treated before beginning treatment exactly. Therefore, the therapeutic agent can be any suitable therapeutic agent, such as, for example, therapeutic agents used to treat any cancer of the brain, lung (e.g., small cell and non-small cell), ovary, breast, prostate, and colon, as well as carcinomas and sarcomas, examples of which can be found in the Physicians' Desk Reference (1998) and elsewhere. For example, in combination with tumor necrosis factor (TNF) treatment of a tumor, a regulatable promoter that is specific for the given tissue, e.g., the tumor, and a marker agent allows a clinician to see exactly which cells are cancerous. Once it is determined which cells to target, any suitable additional therapy, such as radiation therapy or an additional anti-tumor agent, can then be applied only to those affected cells. There are many other suitable applications where imaging would be useful.

### EXAMPLES

The following examples further illustrate the present invention but, of course, should not be construed as in any way limiting its scope.

### Example 1

This example describes the production of a pseudo-receptor for constructing the cell of the present inventive system. Specifically, the exemplary pseudo-receptor includes a binding domain from a single-chain antibody recognizing HA.

Anti-HA ScFv was constructed as an N-Term-VL-VH fusion protein. RT-PCR was performed on RNA obtained from hybridomas producing HA antibodies using primers specific for the κ- or γ2β-terminus and the C-terminus of the VL and VH genes (see Gilliland et al., Tissue Antigens, 47, 1-20 (1996)). After sequencing the resulting PCR products, specific oligonucleotides were designed to amplify the VL-VH fusion in a second round of PCR. The final PCR product was cloned to create the pCANTAB5E(HA) plasmid, described in International Patent Application WO 98/54346, for production of anti HA ScFv in E. coli. The expressed protein has a C-terminal E peptide for detection of binding to HA-tagged penton base via Western analysis of ELISA assay. Upon transformation of bacterial cells with the pCANTAB5E(HA) plasmid, Western analysis using an antibody recognizing the E peptide revealed a protein of the expected size.

To determine whether the anti-HA ScFv was functional, it was used in protein A immunoprecipitation assays using adenoviral coat proteins (recombinant penton base) containing the HA epitope. The anti-HA ScFv was able to precipitate HA-containing penton base proteins. These results indicate the successful construction of the extracellular portion of a pseudo-receptor for binding an adenovirus having a non-native ligand (i.e., HA).

To create an entire anti-HA pseudo-receptor, the anti-HA ScFv was cloned into the pSCHAHK plasmid in which the HA had been removed to create the pScFGHA plasmid, described in International Patent Application WO 98/54346. This plasmid will produce an anti-HA pseudo-receptor able to bind gene transfer vectors, e.g., recombinant adenoviruses, having the HA epitope.

### Example 2

This example demonstrates the increased efficiency of viral infection of host cells of the present inventive system compared to cells not comprising a non-native cell-surface receptor.

Adenoviral vectors comprising the hemagluttenin (HA) tag incorporated into the adenoviral coat protein were generated. One clone was generated such that binding to CAR was ablated (AdL.F*). A clone also was generated such that binding to αᵥ integrin via the penton based was ablated (AdL.PB*). An additional clone was generated such that native binding to CAR and αᵥ integrin was ablated (AdL.PB*F*). Each vector clone contained the luciferase reporter gene driven by the CMV promoter.

Two types of melanoma tumors, B16F0 tumors expressing (B16F0-HA) and not expressing (B16F0) the single-chain antibody directed to HA, a non-native cell-surface receptor, were grown in nude mice. Approximately 10¹⁰ particles of AdL.F,* AdL.PB*F*, and adenoviral vector containing the luciferase gene but not can HA tag (ADL) were administered to each tumor via intratumoral injection. Transduction was quantified via luciferase assay. Transduction of tumors bearing the non-native receptor with AdL and AdL.F* was slightly greater than tumors not comprising the non-native receptor. However, the transduction of B16F0-HA tumors expressing the non-native receptor with AdL.PB*F* was approximately 40-fold greater than transduction of B16F0 tumors not expressing the non-native receptor.

This example demonstrates the ability of the gene transfer vector of the system of present inventive methods to transduce cells of the system more efficiently that cells not comprising the non-native receptor.

### Example 3

This example illustrates the reduced transduction of non-targeted cells by adenoviral vectors comprising recombinant coat proteins comprising a non-native ligand, wherein the non-native ligand preferentially binds αvβ3 integrin.

Adenoviral vectors comprising adenoviral coat proteins lacking fiber binding to CAR (AdL*) and adenoviral vectors comprising adenoviral proteins lacking binding to CAR and penton base-binding to integrins and comprising a ligand that binds αvβ3 integrin (AdL**αv) were constructed. Adenoviral vectors with unmodified coat proteins served as control vectors (AdL). Each vector comprised the luciferase gene.

Equal particle numbers of AdL, AdL*, or AdL**αv were injected into the peritoneal cavity of mice. Animals were sacrificed one day following injection to allow harvesting of the liver, spleen, and kidney, which were assayed for luciferase activity.

Luciferase activity, which is indicative of transduction efficiency, was dramatically reduced (100-fold) in the liver of mice injected with AdL* or AdL**αv compared to liver of mice injected with AdL. Transduction of the kidney and spleen by AdL**αv was reduced by over 100-fold and 10-fold, respectively, compared to those organs of mice injected with AdL.

This example illustrates the ability of an adenoviral vector comprising the present inventive recombinant adenoviral coat protein to avoid transduction of non-targeted cells.

### Example 4

This example illustrates the increased half-life of an adenoviral vector comprising an adenoviral coat protein comprising a non-native ligand, wherein the non-native ligand preferentially binds to a αvβ3 integrin.

Adenoviral vectors comprising adenoviral coat proteins lacking fiber-binding to CAR and penton base-binding to integrins and comprising a ligand specific for αvβ3 integrin (SEQ ID NO: 3) inserted into the HI loop of the fiber protein (AdL.**αv) was constructed using routine techniques and as described herein. To ensure that the adenoviral coat protein could enhance transduction of cells displaying αvβ3 integrin, a panel of cells was infected with the adenoviral vector. A selective increase in transduction was observed for those cell lines that were reported to express αvβ3 integrin.

The kinetics of vector clearance from the blood stream for the tropism-modified vectors were determined as compared to a vector with unmodified tropism (AdL). C3H or C3H-Rag2 mice were injected intrajugularly with 3×10¹⁰ particles of AdL.**αv, AdL (CAR binding, penton base binds to integrins), AdL* (CAR binding ablated, penton base binds to integrins), AdL+ (CAR binding, penton base binding to integrins ablated), or AdL** (CAR binding ablated, penton binding to integrins ablated). At 10 and 60 minutes post-injection, blood serum samples were taken from the mice. These serum samples were assayed to determine the number of vector particles present in the blood. Surprisingly, the AdL.**αv vector has a longer circulating time in the blood compared to all other tested adenoviral constructs. For instance, at 60 minutes post-injection, approximately 10-fold more AdL.**αv vector was detected in the blood stream compared to other vectors administered.

This example demonstrates the ability of an adenoviral vector comprising an adenoviral coat protein of the present invention to remain in blood circulation longer than adenoviral vectors comprising an adenoviral coat protein not comprising a non-native ligand.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Of course, variations of those preferred embodiments will become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. A recombinant adenoviral coat protein comprising a non-native ligand, wherein the non-native ligand binds to a substrate selected from the group of substrates consisting of melanocortin receptor (MC1), αv integrins, αvβ3 integrin, αvβ6 integrin, α4 integrins, α5 integrins, α6 integrins, α9 integrins, CD13, melanoma proteoglycan, membrane dipeptidase (MDP), TAG72 antigen, an antigen binding site of a surface immunoglobulin receptor of B-cell lymphomas, type I interleukin I (IL-1) receptor, human immunodeficiency virus type 1 (HIV-1) envelope glycoprotein (gp120), atrial natriuretic peptide (ANP) receptor, erythropoietin (EPO) receptor, thrombopoietin (TPO) receptor, carcino-embryonic antigen (CEA) receptor, EpCAM, CD40, prostate-specific membrane antigen (PSMA), endoglin, epidermal growth factor receptor (EGFR), HER2, and an extracellular matrix component.

2. The recombinant adenoviral coat protein of claim 1, wherein the non-native ligand binds to a substrate selected from the group of substrates consisting of melanocortin receptor (MC1), α5 integrins, α6 integrins, α9 integrins, CD13, melanoma proteoglycan, membrane dipeptidase (MDP), TAG72 antigen, an antigen binding site of a surface immunoglobulin receptor of B-cell lymphomas, type I interleukin 1 (IL-1) receptor, human immunodeficiency virus type 1 (HIV-1) envelope glycoprotein (gp120), atrial natriuretic peptide (ANP) receptor, erythropoietin (EPO) receptor, thrombopoietin (TPO) receptor, carcino-embryonic antigen (CEA) receptor, EpCAM, CD40, prostate-specific membrane antigen (PSMA), endoglin, epidermal growth factor receptor (EGFR), HER2, and an extracellular matrix component.

3. The recombinant adenoviral coat protein of claim 1, wherein the non-native ligand binds to a substrate selected from the group of substrates consisting of melanocortin receptor (MC1), αv integrins, αvβ6 integrin, α4 integrins, α5 integrins, α6 integrins, α9 integrins, CD13, melanoma proteoglycan, membrane dipeptidase (MDP), TAG72 antigen, an antigen binding site of a surface immunoglobulin receptor of B-cell lymphomas, type I interleukin I (IL-1) receptor, human immunodeficiency virus type 1 (HIV-1) envelope glycoprotein (gp120), atrial natriuretic peptide (ANP) receptor, erythropoietin (EPO) receptor, thrombopoietin (TPO) receptor, and carcino-embryonic antigen (CEA) receptor.

4. The recombinant adenoviral coat protein of claim 3, wherein the ligand comprises a sequence of amino acids selected from the group of sequences consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31.

5. The recombinant adenoviral coat protein of claim 1, wherein the non-native ligand is conjugated to a fiber protein.

6. The recombinant adenoviral coat protein of claim 1, wherein the non-native ligand is conjugated to a penton base protein.

7. The recombinant adenoviral coat protein of claim 1, wherein the non-native ligand is conjugated to a hexon protein.

8. The recombinant adenoviral coat protein of claim 1, wherein the non-native ligand is conjugated to protein IX, VI, or IIIa.

9. The recombinant adenoviral coat protein of claim 1, wherein an adenoviral vector comprising the recombinant adenoviral coat protein lacks native binding.

10. A nucleic acid encoding a recombinant adenoviral coat protein of claim 1.

11. An adenoviral vector comprising the recombinant adenoviral coat protein of claim 1.

12. The adenoviral vector of claim 11, wherein the adenoviral vector comprises a non-native nucleic acid for transcription.

13. A recombinant coat protein comprising a non-native ligand, wherein the non-native ligand binds to a substrate selected from the group of substrates consisting of α4 integrins, αv integrins, αvβ3 integrin, and αvβ6 integrin, and wherein an adenoviral vector comprising the recombinant adenoviral coat protein lacks native binding to coxsackievirus and adenovirus receptor (CAR).

14. The recombinant adenoviral coat protein of claim 13, wherein the non-native ligand preferentially binds αvβ3 integrin.

15. The recombinant adenoviral coat protein of claim 13, wherein the non-native ligand is conjugated to a fiber protein.

16. The recombinant adenoviral coat protein of claim 13, wherein the non-native ligand is conjugated to a penton base protein.

17. A nucleic acid encoding a recombinant adenoviral coat protein of claim 13.

18. An adenoviral vector comprising the recombinant adenoviral coat protein of claim 13.

19. The adenoviral vector of claim 18, which is replication competent.

20. The adenoviral vector of claim 18, wherein the adenoviral vector comprises a non-native nucleic acid for transcription.

21. An adenoviral vector comprising the recombinant adenoviral coat protein of claim 15.

22. The adenoviral vector of claim 21, which is replication competent.

23. The adenoviral vector of claim 21, wherein the adenoviral vector comprises a non-native nucleic acid for transcription.

24. A recombinant coat protein comprising a non-native ligand and a non-native amino acid sequence, wherein the non-native ligand binds to a matrix metalloproteinase (MMP).

25. The recombinant adenoviral coat protein of claim 24, wherein the non-native ligand comprises SEQ ID NO:12.

26. An adenoviral vector comprising a modification and a fiber protein that lacks native binding, wherein the modified adenoviral vector elicits less reticulo-endothelial system (RES) clearance in a host animal than a corresponding wild-type adenovirus.

27. The adenoviral vector of claim 26, wherein the adenoviral vector lacks a native glycosylation or phosphorylation site.

28. The adenoviral vector of claim 26, wherein the adenoviral vector is functionally-linked to a molecule that masks the adenoviral vector from recognition by the RES or neutralizing antibodies.

29. The adenoviral vector of claim 26, wherein the adenoviral vector is functionally-linked to a lipid derivative of polyethylene glycol having a primary amine group, an epoxy group, or a diacylglycerol group.

30. The adenoviral vector of claim 29, wherein the adenoviral vector is conjugated to a lipid derivative of polyethylene glycol having a primary amine group, an epoxy group, or a diacylglycerol group.

31. The adenoviral vector of claim 26, wherein the adenoviral vector comprises one or more chimeric adenoviral coat proteins.

32. The adenoviral vector of claim 31, wherein the chimeric adenoviral coat protein is a hexon, penton base, or fiber protein.

33. The adenoviral vector of claim 31, wherein the chimeric adenoviral coat protein is protein IX, protein VI, or protein IIIa.

34. A system comprising (i) a cell having a non-native cell-surface receptor, and (ii) a virus having a non-native ligand, wherein the cell is in vivo and the non-native ligand of the virus binds the non-native cell-surface receptor of the cell.

35. The system of claim 34, wherein the virus is an adenovirus.

36. The system of claim 35, wherein a transgenic test or laboratory animal comprises the cell having a non-native cell-surface receptor.

37. The system of claim 35, wherein the cell having a non-native cell-surface receptor is localized within specific tissue of the transgenic test or laboratory animal.

38. The system of claim 37, wherein localization of the cell having a non-native cell-surface receptor to specific tissue within the transgenic test or laboratory animal is through tissue-specific regulation of the non-native cell-surface receptor.

39. The system of claim 34, wherein the cell replicates the virus upon binding of the ligand to the non-native cell-surface receptor, and internalization of the virus.

40. The system of claim 35, wherein the non-native cell-surface receptor is a non-adenoviral receptor, which binds a substrate other than a native adenoviral ligand.

41. The system of claim 35, wherein the non-native cell-surface receptor is a non-adenoviral receptor and the non-native ligand of the virus binds to the non-adenovirus receptor.

42. The system of claim 34, wherein the non-native cell-surface receptor is a protein comprising a domain derived from an immunoglobulin.

43. A method of propagating a virus comprising infecting the cell of claim 34 with a virus, maintaining the cell, and recovering the virus produced within the cell.

44. A method of propagating a virus comprising infecting the cell of claim 35 with a virus, maintaining the cell, and recovering the virus produced within the cell.

45. A method of propagating a virus, wherein the method comprises (a) infecting a cell having a non-adenovirus cell-surface receptor with a virus having a non-native ligand, wherein the non-native ligand of the virus binds the non-adenovirus cell-surface receptor, (b) maintaining the cell, and (c) recovering the virus produced within the cell.

46. A method of isolating a nucleic acid encoding a product comprising a desired property comprising
(a) infecting cells with a library of gene transfer vectors, wherein each cell has a non-native cell-surface receptor, and wherein each gene transfer vector comprises (i) a ligand that binds the non-native cell-surface receptor of the cell and (ii) a nucleic acid encoding a product comprising a potentially desired property,
(b) assaying the cells comprising the library of gene transfer vectors for a desired property, and
(c) isolating the gene transfer vector comprising the nucleic acid encoding the product comprising the desired property.

47. A method of identifying functionally related coding sequences comprising
(a) infecting cells with a library of gene transfer vectors, wherein each cell has a non-native cell-surface receptor and wherein each gene transfer vector comprises (i) a ligand that binds the non-native cell-surface receptor of the cell, (ii) a first heterologous DNA encoding a first gene product, wherein the first DNA is common to each gene transfer vector, and (iii) a second heterologous DNA encoding an second gene product, wherein the second DNA varies between the gene transfer vectors, and
(b) comparing the activity of the gene products encoded by the gene transfer vectors with the activity of the first gene product encoded by a gene transfer vector comprising the first heterologous DNA but not comprising the second heterologous DNA.

48. A method of assaying for gene function comprising (a) infecting a cell having a cell-surface receptor that is overexpressed in the cell with a gene transfer vector comprising a ligand that binds the cell-surface receptor of the cell, (b) maintaining the cell, and (c) assaying the cell for alterations in physiology.

49. A method of isolating a nucleic acid encoding a product comprising a desired property comprising
(a) infecting cells with a library of gene transfer vectors, wherein each cell has a cell-surface receptor that is overexpressed in the cell, and wherein each gene transfer vector comprises (i) a ligand that binds the cell-surface receptor of the cell and (ii) a nucleic acid encoding a product comprising a potentially desired property,
(b) assaying the cells comprising the library of gene transfer vectors for a desired property, and
(c) isolating the gene transfer vector comprising the nucleic acid encoding the product comprising the desired property.

50. The method of claim 47, wherein the gene transfer vector is a virus.

51. The method of claim 50, wherein the virus is an adenovirus.

52. The method of claim 47, wherein an animal comprises the cell(s) having a non-native cell-surface receptor.

53. The method of claim 48, wherein a population of cells comprises the cell(s) having the non-native cell-surface receptor and cells not having the non-native cell-surface receptor.

54. The method of claim 53, wherein the gene transfer vector does not bind to the cells not having the non-native cell-surface receptor.

55. The method of claim 48, wherein the gene transfer vector is a virus.

56. The method of claim 55, wherein the virus is an adenovirus.

57. The method of claim 48, wherein the cell(s) having a non-native cell-surface receptor is localized within specific tissue of the animal.

58. The method of claim 46, wherein the library of gene transfer vectors comprise at least one additional nucleic acid encoding a different product, wherein the additional nucleic acid sequence is common to each gene transfer vector.

59. A method of controlled gene expression comprising administering to an animal a selectively replication competent adenoviral vector having a first non-native nucleic acid, operably linked to a promoter, and a targeting agent.

60. The method of claim 59, wherein the adenoviral vector comprises deletions in the E1a and E1b region of the adenoviral genome of the adenoviral vector.

61. The method of claim 60, wherein the adenoviral vector further comprises a second non-native nucleic acid and wherein the second non-native nucleic acid is for selective replication.

62. The method of claim 61, wherein the second non-native nucleic acid is operably linked to a regulatable promoter or a tissue-specific promoter.

63. The method of claim 62, wherein the adenoviral vector is rendered replication competent upon expression of the second non-native nucleic acid.

64. The method of claim 63, wherein the first non-native nucleic acid is expressed upon replication of the adenoviral vector.

65. A cell-surface receptor comprising a first domain and a second domain, wherein the first domain binds an adenoviral vector having one or more chimeric adenoviral coat proteins and the second domain facilitates internalization of the adenoviral vector into a cell.

66. The cell-surface receptor of claim 65, wherein the cell-surface receptor is a non-native, non-adenovirus cell-surface receptor, and the second domain actively facilitates internalization of the adenoviral vector into the cell.

67. The cell-surface receptor of claim 65, wherein the second domain is a transmembrane domain fused to an internalization domain selected from the group consisting of an LDL cytoplasmic domain and an αvβ5 integrin cytoplasmic domain.

68. A non-native, non-adenovirus cell-surface receptor comprising a first domain and a second domain, wherein the first domain binds an adenoviral vector having one or more chimeric adenoviral coat proteins and the second domain is a glycerol-phosphate-inositol linkage.

69. A cell comprising the cell-surface receptor of claim 65.

70. A method of therapy comprising administering to an animal an adenoviral vector having (i) a first non-native nucleic acid, (ii) a second non-native nucleic acid, and (iii) a targeting agent, wherein the first non-native nucleic acid encodes a therapeutic agent and the second non-native nucleic acid encodes an agent that facilitates imaging.

71. The method of claim 70, wherein the therapeutic agent is an anti-tumor agent.

72. The method of claim 71, wherein the anti-tumor agent is tumor necrosis factor (TNF).

73. The adenoviral vector of claim 26, wherein the adenoviral vector further comprises a penton protein that lacks native binding.

74. An adenoviral vector comprising a fiber protein that lacks native binding, wherein the adenoviral vector is functionally-linked to polyethylene glycol (PEG).


SEQUENCE LISTING (31 sequences)

SEQ ID NO: 1 - protein, 13 aa, Homo sapiens
SSIISHFRWGLCN

SEQ ID NO: 2 - protein, 5 aa, Homo sapiens
CRGDC

SEQ ID NO: 3 - protein, 9 aa, Homo sapiens
CDCRGDCFC

SEQ ID NO: 4 - protein, 12 aa, Homo sapiens
RTDLDSLRTYTL

SEQ ID NO: 5 - protein, 6 aa, Homo sapiens
CWLDVC

SEQ ID NO: 6 - protein, 9 aa, Homo sapiens
CRRETAWAC

SEQ ID NO: 7 - protein, 15 aa, Homo sapiens
VSWFSRHRYSPFAVS

SEQ ID NO: 8 - protein, 12 aa, Homo sapiens
PLAEIDGIELTY

SEQ ID NO: 9 - protein, 13 aa, Homo sapiens
CNGRCVSGCAGRC

SEQ ID NO: 10 - protein, 10 aa, Homo sapiens
TAASGVRSMH

SEQ ID NO: 11 - protein, 10 aa, Homo sapiens
LTLRWVGLMS

SEQ ID NO: 12 - protein, 10 aa, Homo sapiens
CTTHWGFTLC

SEQ ID NO: 13 - protein, 13 aa, Homo sapiens
CGFECVRQCPERC

SEQ ID NO: 14 - protein, 7 aa, Homo sapiens
YSGKWGW

SEQ ID NO: 15 - protein, 13 aa, Homo sapiens
CVALCREACGEGC

SEQ ID NO: 16 - protein, 9 aa, Homo sapiens
SWCEPGWCR

SEQ ID NO: 17 - protein, 10 aa, Homo sapiens
HYVSIELPDH

SEQ ID NO: 18 - protein, 10 aa, Homo sapiens
GAARTLRFGA

SEQ ID NO: 19 - protein, 8 aa, Homo sapiens
KPWWYSRV

SEQ ID NO: 20 - protein, 8 aa, Homo sapiens
DWAIWSKR

SEQ ID NO: 21 - protein, 9 aa, Homo sapiens
YSFEDLYRR

SEQ ID NO: 22 - protein, 21 aa, Homo sapiens
ETPFTWEESNAYYWQPYALPL

SEQ ID NO: 23 - protein, 20 aa, Homo sapiens
TANVSSFEWTPGYQPYALPL

SEQ ID NO: 24 - protein, 20 aa, Homo sapiens
DGYDRWQSGERYWQPYALPL

SEQ ID NO: 25 - protein, 12 aa, Homo sapiens
RINNIPWSEAMM

SEQ ID NO: 26 - protein, 12 aa, Homo sapiens
TSPYEDWQTYLM

SEQ ID NO: 27 - protein, 12 aa, Homo sapiens
SQRWTALWQWIG

SEQ ID NO: 28 - protein, 7 aa, Homo sapiens
ASSLNIA

SEQ ID NO: 29 - protein, 15 aa, Homo sapiens
MCHFGGRMDRISCYR

SEQ ID NO: 30 - protein, 18 aa, Homo sapiens
DREGCRRGWVGQCKAWFN

SEQ ID NO: 31 - protein, 14 aa, Homo sapiens
IEGPTLRQWLAARA